(19) United States
(12) Patent Application Publication
(10) Pub. No.: US 2013/0245557 A1
(43) Pub. Date: Sep. 19, 2013

(54) Medical Lanyard

(71) Applicant: Paul T. Brasington, Box Springs, GA (US)

(72) Inventor: Paul T. Brasington, Box Springs, GA (US)

(21) Appl. No.: 13/422,661

(22) Filed: Mar. 16, 2012

(51) Int. Cl. A61M 25/02

(52) U.S. Cl. 604/179; 604/174

(57) ABSTRACT

A medical lanyard for securing a feeding tube to a patient, increasing patient maneuverability, and protecting against unintended removal of the feeding tube is provided. The medical lanyards are worn around a patient's neck or clipped to the patient's clothing. The medical lanyard securely suspends a feeding tube port connector and the corresponding feeding tube while providing for easy disconnect of the feeding tube port connector/feeding tube combination from either the necklace or the clip, or from the nutrient supply. The medical lanyards also provide for protection of the feeding tube stomach insertion point so that it is not compromised when tension is applied between the feeding tube port connector/feeding tube combination and either the remainder of the medical lanyard or the nutrient supply.

### CROSS-REFERENCE TO RELATED APPLICATIONS

Not Applicable

### BACKGROUND

1. Field of Invention

This invention pertains to medical feeding tube devices. More particularly, this invention pertains to medical lanyards for securing a feeding tube port connector in a position for the reduction of patient discomfort related to feeding tubes and tensions associated therewith.

2. Description of the Related Art

Feeding tube devices are commonly used to provide nutrients to patients that are incapable of consuming nutrients by swallowing food. Feeding tube use may be temporary for the treatment of acute conditions or lifelong for chronic conditions or disabilities.

Gastric feeding tubes are inserted through a small incision in the abdomen into the stomach. One type of gastric feeding tube is a percutaneous endoscopic gastrostomy (PEG) tube. Gastric feeding tubes such as PEG tubes are often used for long-term enteral nutrition.

Gastric feeding tubes have typically been secured to the patient via an adhesive or bandage. The feeding tube extends from the patient's body and is attached to a feeding tube port connector. Another tube connects the feeding tube port connector to the nutrient supply package. In such circumstances, the feeding tube is vulnerable to being pulled out of the abdomen due to inadvertent removal or relocation of the nutrient supply. Additionally, temporarily disconnecting the nutrient supply leaves a lengthy feeding tube which must be accounted for when not attached.

In other situations, gastric feeding tubes have been secured to patients and/or patient clothing via safety pins, belts, and necklace type tethers that are detachable from the feeding tube. While reducing the reliance on an adhesive for maintaining the feeding tube in place, the adjustability of such methods has been less than desirable.

### BRIEF SUMMARY

According to one embodiment of the present invention, a medical lanyard for securing a feeding tube port connector in a position to reduce patient discomfort related to feeding tubes and tensions associated therewith is disclosed. The medical lanyard provides increased patient maneuverability, and protection against unintended removal of the feeding tube.

The medical lanyards disclosed herein are worn around a patient's neck or clipped to the patient's clothing. The medical lanyard securely suspends a feeding tube port connector and the corresponding feeding tube while providing for easy disconnect of the feeding tube/feeding tube port connector combination from either the necklace or the clip, or from the nutrient supply. The disclosed medical lanyards also provide for protection of the feeding tube stomach insertion point so that it is not compromised when tension is applied between the feeding tube/feeding port combination and either the remainder of the medical lanyard or the nutrient supply.

In one embodiment, a medical lanyard for securing a feeding tube port connector in a position to reduce patient discomfort due to feeding tube tension is disclosed. The medical lanyard includes (1) a first strap assembly including a first loop sized to fit around a patient neck, and an adjustment mechanism to adjust size of the first loop, (2) a second strap assembly including a second loop, and an O-ring sized for the feeding tube port connector to fit within the O-ring, wherein the second loop extends through the O-ring, and (3) a breakaway fastener for detachably connecting the second strap assembly to the first strap assembly, the breakaway fastener including a first portion in concert with the first loop, and a second portion in concert with the second loop, wherein the breakaway fastener is operable to detach the first portion and the second portion from each other upon an increased tension in the second strap assembly, and whereby when the second strap assembly is connected to the first strap assembly, the adjustment mechanism provides for adjusting the size of the first loop to vary length of the medical lanyard to locate the O-ring in proximity of a feeding tube entry point so that feeding tube tension is reduced.

In one embodiment the breakaway fastener further provides means for rotating the first portion and the second portion in relation to each other to provide a twisting operation between the first strap assembly and the second strap assembly. In one embodiment, the rotating means provides for lateral adjustment of the second strap assembly in relation to the first strap assembly.

Other systems, methods, features and advantages of the present invention will be or become apparent to one with skill in the art upon examination of the following drawings and detailed description. It is intended that all such additional systems, methods, features and advantages be included within this description and be within the scope of the present disclosure.

### BRIEF DESCRIPTION OF THE DRAWINGS

The above-mentioned features will become more clearly understood from the following detailed description read together with the drawings in which:

FIG. 1 is a prior art illustration of a feeding tube port connector administered to a patient.

FIG. 2 is an embodiment of a medical lanyard for securing a feeding tube port connector to the patient.

FIG. 3 is an exploded view of the medical lanyard of FIG. 2.

FIG. 4 is an alternative embodiment of a medical lanyard for securing a feeding tube port connector to a patient via a clip.

FIG. 5 is an exploded view of the medical lanyard of FIG. 4.

FIG. 6 is an illustration of an O-ring for securing a feeding tube port connector.

FIG. 7 is an illustration of one embodiment of a medical lanyard securing a feeding tube port connector.

FIG. 8 is an illustration of another embodiment of a medical lanyard securing a feeding tube port connector.

### DETAILED DESCRIPTION

A medical lanyard is disclosed for securing a feeding tube port connector in position to reduce patient discomfort related to feeding tubes and tensions associated therewith. The medical lanyard also provides increased patient maneuverability, and protection against unintended removal of the feeding tube.

In particular, the medical lanyard secures a feeding tube port connector or other similar type connector for reduction of patient discomfort. For example, patient discomfort or even pain can occur at the feeding tube insertion point when a nutrient source or supply connected to a feeding tube is moved and causes a pulling or tension on the feeding tube. The present invention also reduces the likelihood of the feeding tube being inadvertently pulled from the stomach.

The medical lanyards disclosed herein are worn around a patient's neck or clipped to the patient's clothing. The medical lanyard securely suspends a feeding tube port connector and the corresponding feeding tube while providing for easy disconnect of the feeding tube/feeding tube port connector combination from either the necklace or the clip, or from the nutrient supply. The disclosed medical lanyards also provide for protection of the stomach insertion point of the feeding tube so that it is not compromised when tension is applied between the feeding tube/feeding tube port combination and either the remainder of the medical lanyard or the nutrient supply.

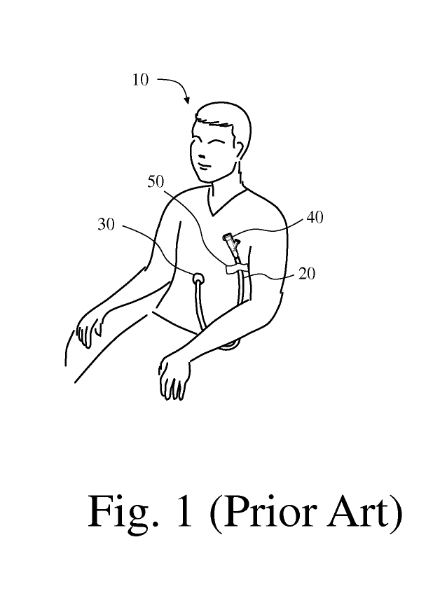

FIG. 1 is an illustration of a conventional feeding tube 20 administered to a patient 10. The feeding tube 20 extends from one end at the stomach insertion point 30 to a feeding tube port connector 40. The feeding tube 20 is often secured to the patient 10 at or near the feeding tube port connector 40 end via an adhesive bandage 50.

Feeding tubes 20 are commonly used to provide nutrients to patients 10 that are incapable of consuming nutrients by swallowing food. Feeding tube use may be temporary during the treatment of acute conditions or its use may be lifelong for chronic conditions or disabilities. Gastric feeding tubes 20 are inserted through a small incision in the abdomen and into the stomach. This small incision is the stomach insertion point 30. One type of gastric feeding tube 20 is a percutaneous endoscopic gastrostomy (PEG) tube. Gastric feeding tubes 20 such as PEG tubes are often used for long-term enteral nutrition.

Gastric feeding tubes 20 have typically been secured to the patient 10 via an adhesive (or bandage) 50 that is applied to the feeding tube 20 and to a location on the patient's body in a manner intended to keep the feeding tube 20 and the feeding tube port connector 40 in place. This application of an adhesive 50 is often located near the feeding tube port connector 40 to prevent the feeding tube port connector 40 and/or nutrient supply from dangling. In such circumstances, the feeding tube 20 is vulnerable to being pulled from the abdomen due to any increased tension on the feeding tube 20. Some events that increase tension on the feeding tube 20 include sudden patient movement, patient twisting, patient turning, inadvertent removal of the nutrient supply, and other events that directly or indirectly pull or snatch the feeding tube 20.

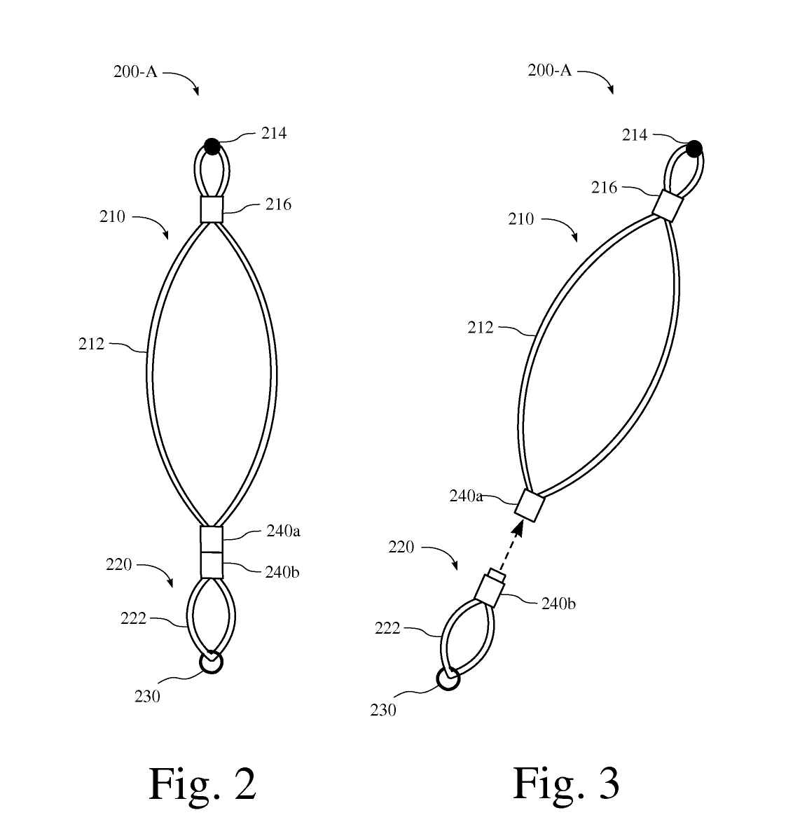

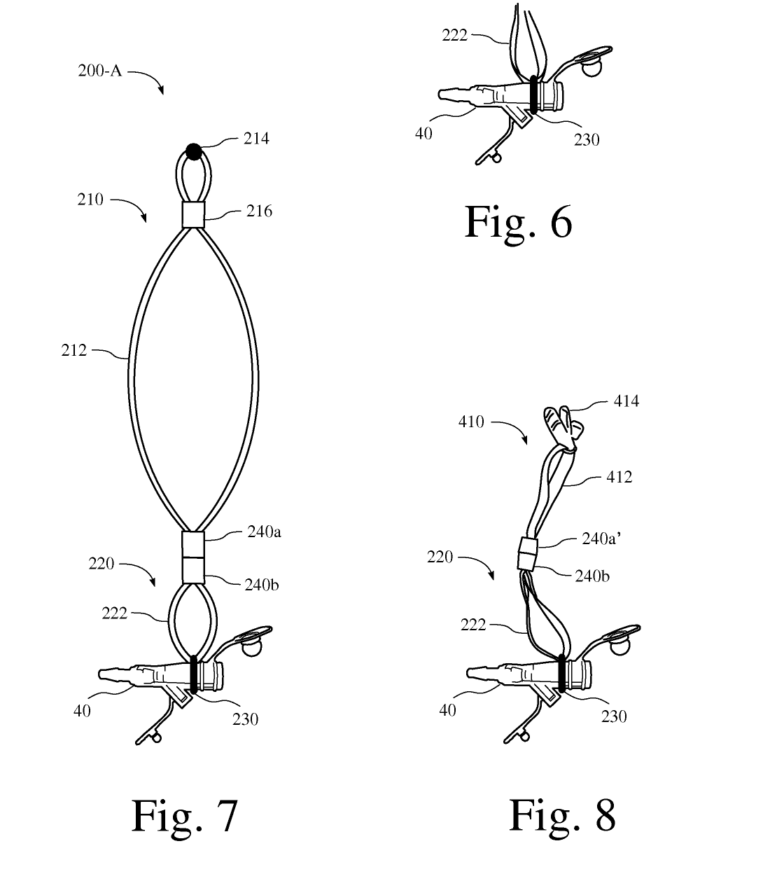

FIG. 2 is an embodiment of a medical lanyard 200-A for securing a feeding tube port connector 40 (see FIG. 6) in position to reduce patient discomfort related to feeding tubes 20 and tensions associated with their use. FIG. 3 is an exploded view of the medical lanyard 200-A. The medical lanyard 200-A is of a necklace type and is placed around the neck for securing the feeding tube port connector 40 to the patient 10. The medical lanyard 200-A includes a loop assembly 210, an attachment assembly 220, and a fastener 240. In the illustrated embodiment, the fastener 240 is a breakaway fastener, and includes a female fastener 240a portion and a male fastener portion 240b. The female fastener 240a and the male fastener 240b together provide for removably securing the attachment assembly 220 to the loop assembly 210 of the medical lanyard 220-A.

The loop assembly 210 is a loop portion, or strap assembly, that includes a loop strap 212, a stop 214, an adjustment mechanism 216, and a female fastener 240a. The loop strap 212 is typically a cord, rope, string, or similar material. Those of skill in the art will readily appreciate that the loop strap 212 can be made from many materials, such as soft fabric, polyester, nylon, satin, silk, or polyethylene terephthalate (PET), among others. The loop strap 212 is a loop having sufficient length when stretched that it fits around the neck of a patient 10 while also providing enough slack that the attachment assembly 220 is positioned in the stomach area of the abdomen at one end, and enough slack for adjustment at the other end of the stretched out loop. In one embodiment, the loop strap 212 is one piece of material. In such an embodiment, the loop strap 212 extends through the stop 214 so that the stop 214 forms an end to the two sections of the loop strap 212. The stop 214 provides a restraint so that the adjustment mechanism 216 does not slide off the loop strap 212. In one embodiment, the stop 214 is round or cylindrical and includes a hole through which the loop strap 212 extends. In the illustrated embodiment, the stop 214 is a bead with a hole through which the loop strap 212 extends. In other embodiments, the stop 214 is any type material or mechanism to prevent the adjustment mechanism 216 from sliding past the end of the loop assembly 210. In one embodiment, two ends of the loop strap 212 are attached to a female fastener 240a to form an opposite end to the stop 214 end of the loop strap 212. That is, the loop strap 212 is essentially folded in half with a stop 214 at one end and with a female fastener 240a at the opposite end. In other embodiments, the stop 214 is any mechanism for defining or separating the strap into two portions. In another embodiment, the loop strap 212 is a single piece of circular material having no ends. In such an embodiment, the fastener 240 is of a type suitable for enclosing a portion of the loop strap 212 without regard for “threading” the loop strap 212 through an opening. For example, one such fastener 240 clamps to the loop strap 212. Those of skill in the art will readily appreciate that other such fasteners 240 are also suitable for use with such an embodiment.

An adjustment mechanism 216, or slide, is attached to the loop strap 212 so that each section of the loop strap 212, separated by the stop 214, extends through the adjustment mechanism 216. That is, the stop 214 divides the loop strap 212 into two portions. The adjustment mechanism 216 divides the loop assembly 210 into two loops. The loop adjacent the fastener 240 is the larger of the two and is placed around the neck of the patient 10. The adjustment mechanism is adjusted toward or away from the stop 214 to adjust the effective length of the medical lanyard 200-A. Sliding the adjustment mechanism 216 toward the stop 214 increases the size of the loop adjacent the fastener 240, effectively lengthening the medical lanyard 200-A. Sliding the adjustment mechanism away from the stop 214 decreases the loop adjacent the fastener 240, effectively reducing the length of the medical lanyard 200-A. Lengthening the medical lanyard 200-A (increasing the loop adjacent the fastener 240) effectively moves the position of the feeding tube port connector 40 lower (with the patient in a standing or sitting position) on the patient's torso, that is distal to the patient's head.

The adjustment mechanism 216 can be any type device having an opening through which the separate sections of the loop strap 212 extend together. The adjustment mechanism 216 provides for the separate sections of the loop strap 212 to fit securely and together through the adjustment mechanism 216 so that the adjustment mechanism 216 does not slide inadvertently. That is, the separate sections of the loop strap 212 fit snugly within the adjustment mechanism 216 so that the adjustment mechanism 216 does not slide on its own. The adjustment mechanism 216 has the capability to slide so that the respective loops formed by the loop strap 212 increase or decrease in size depending upon the direction of motion.

The attachment assembly 220 includes an attachment strap 222, a male fastener 240b, and an O-ring 230. Similar to the loop strap 212 discussed above, the attachment strap 222 is typically a cord, rope, string, or similar material, and forms a small loop that includes an O-ring 230. Those of skill in the art will readily appreciate that the attachment strap 222 can be made from many materials, such as soft fabric, polyester, nylon, satin, silk, or polyethylene terephthalate (PET), among others. In the illustrated embodiment, the attachment strap 222 extends through the O-ring 230. In this way the O-ring “hangs” from the attachment strap 222 during typical usage. The small loop combines with the O-ring 230 to support a feeding tube port connector 40, such as a Y-port connector (see FIG. 6 below).

In one embodiment, the O-ring 230 is rubber. In such an embodiment, the O-ring 230 is flexible enough to allow for insertion and removal of a feeding tube port connector 40, while also providing enough snugness to maintain the feeding tube port connector 40 securely in place. It will be appreciated by those of skill in the art that the O-ring 230 may be many other materials providing characteristics of flexibility for insertion and removal of the feeding tube port connector 40 and also providing for maintaining the feeding tube port connector 40 securely in position.

The male fastener 240b of the attachment assembly 220 is secured to the female fastener 240a of the loop assembly 210 to form the medical lanyard 200-A. Those of skill in the art will readily appreciate that the male fastener 240b and the female fastener 240a can be reversed without adversely impacting the functionality of the medical lanyard 200-A. That is, the male fastener 240b can be attached to the loop strap 212 of the loop assembly 210, and the female fastener 240a can be attached to the attachment strap 222 of the attachment assembly 220.

The male fastener 240b and the female fastener 240a together form a breakaway type mechanism. That is, when tension is applied to the combination of the medical lanyard 220-A and the feeding tube 20, the breakaway functionality ensures that the stomach insertion point 30 of the feeding tube 20 is not compromised. The breakaway functionality of the fastener 240 also provides for easy detachment of the feeding tube 20, via detaching the attachment assembly 220, during tube cleaning for example.

In other embodiments of the necklace type medical lanyard 220-A, the combination female fastener 240a and male fastener 240b are replaced by other type fasteners 240 that adjoin together to secure the attachment assembly 220 to the loop assembly 210. In various embodiments, the fastener 240 is a breakaway buckle, a breakaway cord, a detachable clasp, or a snap, among others. Those of skill in the art will readily appreciate that there are numerous types of fasteners 240 that are capable of fastening to each other and also to the loop strap 212 and/or the attachment strap 222, while also maintaining breakaway functionality to protect the integrity of the stomach insertion point 30 of the feeding tube 20.

In another embodiment, the fastener 240 includes a rotational functionality. In such an embodiment, the female fastener 240a and the male fastener 240b connect in such a manner that either fastener portion is rotatable with respect to the other fastener portion. This provides a rotational or twisting operation between the loop assembly 210 and the attachment assembly 220. This rotational functionality provides for increased lateral adjustment of the medical lanyard 200-A. For example, rotation of the attachment assembly 220 relative to the loop assembly 210 allows for additional flexibility due to the orientation of the feeding tube 20 or of the nutrient supply.

The medical lanyard 220-A is worn about a patient's neck so that the feeding tube 20 is suspended by the O-ring 230 at the end of the attachment assembly 220.

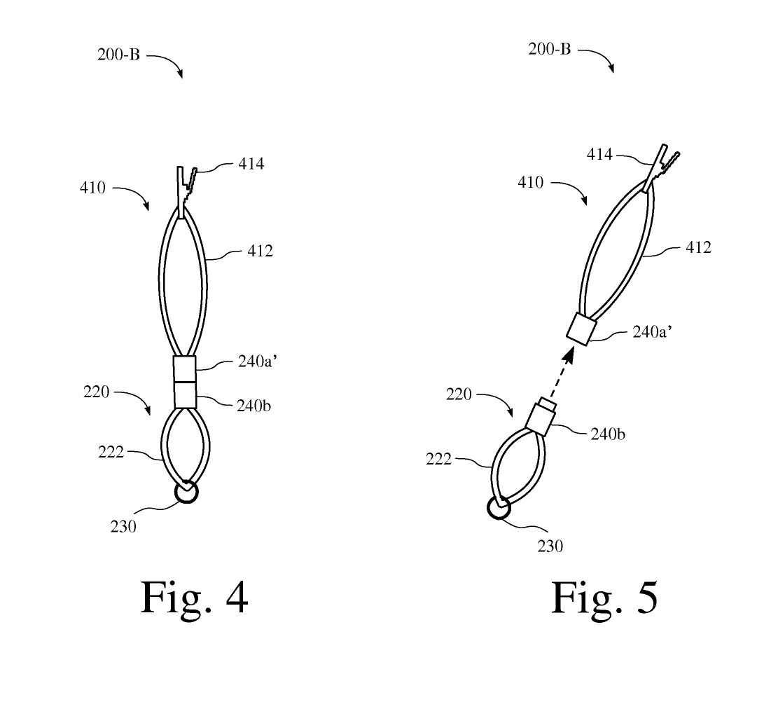

FIG. 4 is an alternative embodiment of a medical lanyard 200-B for securing a feeding tube 20 to a patient 10 and FIG. 5 is an exploded view of the medical lanyard 200-B. The medical lanyard 200-B is a clip type lanyard and is secured via attachment of the clip 414 to an item such as clothing. Alternatively, the clip 414 is connected to other items as desired. The medical lanyard 200-B includes a clip assembly 410 and an attachment assembly 220. The attachment assembly 220 is as described above with reference to the necklace type lanyard.

The clip assembly 410 of the medical lanyard 200-B includes a clip strap 412, a clip 414, and a female fastener 240a′. The clip strap 412 is typically a cord, rope, string, or similar material, suitable for supporting the attachment assembly 220. In one embodiment, the clip strap 412 is one piece of material having two ends. Both ends of the clip strap 412 are attached to the female fastener 240a′. The clip 414 is attached at the midpoint between the ends of the clip strap 412. In this way, the clip 414 separates the clip strap 412 into two separate portions having the clip 414 at one end of the clip assembly 410 and the female fastener 240a′ at the opposite end of the clip assembly 410. The female fastener 240a′ is as described above. In one embodiment, the clip 414 includes an opening through which the clip strap 412 extends. Those of skill in the art will readily appreciate that there are numerous types of clips that include a variety of fasteners and openings for attaching to straps and the like.

In another embodiment, the clip strap 412 is a single piece of circular material having no ends. In such an embodiment, the fastener 240 is of a type suitable for enclosing a portion of the loop strap 212 without regard for “threading” the loop strap 212 through an opening. For example, one such fastener 240 clamps to the clip strap 412. Those of skill in the art will readily appreciate that other such fasteners 240 are also suitable for use with such an embodiment.

The male fastener 240b of the attachment assembly 220 is secured to the female fastener 240a′ of the clip assembly 410 to form the clip type medical lanyard 200-B. As noted above, those of skill in the art will readily appreciate that the male fastener 240b and the female fastener 240a′ can be reversed without adversely impacting the functionality of the medical lanyard 200-B. That is the male fastener 240b can be attached to the clip strap 412 of the clip assembly 410, and the female fastener 240a′ can be attached to the attachment strap 222 of the attachment assembly 220.

As noted above, the male fastener 240b and the female fastener 240a′ together form a breakaway type fastener mechanism. When tension is applied to the combination of the medical lanyard 220-B and the feeding tube 20, the breakaway functionality ensures that the stomach insertion point 30 of the feeding tube 20 is not compromised. The breakaway functionality of the clasps 218′, 224 also provides for easy detachment of the feeding tube 20, via detaching the attachment assembly 220 from the clip assembly 410.

In other embodiments of the clip type medical lanyard 200-B, the combination female fastener 240a′ and male fastener 240b are replaced by other type fasteners 240 that adjoin together to secure the attachment assembly 220 to the clip assembly 410. In various embodiments, the fastener 240 is a breakaway buckle, a breakaway cord, a detachable clasp, or a snap, among others. Those of skill in the art will readily appreciate that there are numerous types of fasteners 240 that are capable of fastening to each other and also to the clip strap 412 and/or the attachment strap 222, while also maintaining breakaway functionality to protect the integrity of the stomach insertion point 30 of the feeding tube 20.

As described above, the fastener 240 includes a rotational functionality. In such an embodiment, the female fastener 240a and the male fastener 240b connect in such a manner that either fastener portion is rotatable with respect to the other fastener portion. This provides a rotational or twisting operation between the clip assembly 410 and the attachment assembly 220. This rotational functionality provides for increased lateral adjustment of the medical lanyard 200-A. For example, rotation of the attachment assembly 220 relative to the clip assembly 410 allows for additional flexibility due to the orientation of the feeding tube 20 or of the nutrient supply.

The clip type medical lanyard 200-B is used by attaching the clip 414 to the clothing worn by the patient 10. In one embodiment the clip 414 is attached to a hospital gown or other covering that is worn by the patient 10. In such uses the clip can be attached in a manner that allows for support of the feeding tube 20 while otherwise minimizing inconvenience to the patient 10.

FIG. 6 is an illustration of an O-ring 230 for securing a feeding tube port connector 40. In the illustrated embodiment the feeding tube port connector 40 is a Y-port connector. The attachment strap 222 extends through the O-ring 230 so that the O-ring 230 “hangs” from the attachment strap 222. The feeding tube port connector 40 fits securely within the O-ring 230.

FIG. 7 is an illustration of one embodiment of a necklace type medical lanyard 200-A securing a feeding tube port connector 40. FIG. 8 is an illustration of another embodiment of a clip type medical lanyard 200-B securing a feeding tube port connector 40. The feeding tube port connector 40 includes a connection point for a feeding tube 20 that is inserted into the stomach, and another connection point for an additional tube and/or nutrient supply.

The attachment assembly 220 secures the feeding tube port connector 40 via the O-ring 230 of the attachment assembly 220. The attachment assembly 220 is connectable to either the loop assembly 210 of the necklace type medical lanyard 220-A, or to the clip assembly 410 of the clip type medical lanyard 220-B. The attachment assembly 220 is easily detachable from and re-attachable to the loop assembly 210 by disconnecting the male fastener 240b from the female fastener 240a. Similarly, the attachment assembly 220 is detachable from and re-attachable to the clip assembly 410 by disconnecting the male fastener 240b from the female fastener 240a′.

Detaching the attachment assembly 220 from the loop assembly 210 or from the clip assembly 410 provides for increased flexibility and maneuverability by the patient.

The medical lanyard 200 also provides support for the feeding tube 20 when not in use. The nutrient supply is disconnected by removing the additional tube from the feeding tube port connector 40. In this way, a shorter feeding tube 20 assembly remains inserted into the stomach of the patient 10. The patient 10 is not burdened with additional tubes extending from the feeding tube port connector 40. The nutrients are removed via disconnecting the nutrient supply and/or tube from the feeding tube port connector 40. The short feeding tube 20 remains in the stomach incision and connected to the feeding tube port connector 40. The feeding tube port connector 40 is secured by the O-ring which hangs from the attachment strap 222 of the attachment assembly 220 which is either connected to the loop assembly 210 of a medical lanyard 200, such as a necklace type medical lanyard 220-A, or to the clip assembly 410 of a clip type medical lanyard 220-B. In this way, the feeding tube port connector 40 remains connected to the feeding tube 20 which remains inserted in the stomach incision even when the nutrients are disconnected. The patient 10 maneuverability is greatly improved by easily disconnecting from additional nutrient supplies and/or tubes.

From the foregoing description, it will be recognized by those skilled in the art that a medical lanyard 200 for securing a feeding tube 20 to a patient while increasing maneuverability and protecting against unwanted removal of the feeding tube 20 has been provided.

The medical lanyards 200 disclosed herein are worn around a patient's neck or clipped to the patient's clothing. The medical lanyard 200 securely suspends a feeding tube port connector 40 and the corresponding feeding tube 20 while providing for easy disconnect of the attachment assembly 220 (feeding tube port connector 40/feeding tube 20 combination) from either the loop assembly 210 (necklace) or the clip assembly 410, or from the nutrient supply and/or other tubes. The disclosed medical lanyards 200 also provide for protection of the feeding tube stomach insertion point 30 so that it is not compromised when tension is applied between the attachment assembly 220 and either the loop assembly 210 or the clip assembly 410, or between the attachment assembly 220 and the nutrient supply and/or other tubes.

In one embodiment, the medical lanyards 200 disclosed herein provide for securing feeding tube port connectors 40 and the corresponding feeding tube 20 in manners that are easier to wear under clothing while increasing comfort for the patient. In one such embodiment, the straps 212, 222, 412 are made from soft fabric to reduce rubbing or pulling of hair. Additionally, the medical lanyards 200 disclosed herein are lightweight and loose fitting for increased comfort. By comparison with the use of adhesives or bandages, the embodiments of medical lanyards 200 disclosed herein are not sticky or painful to remove or disconnect from the patient.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. A medical lanyard for securing a feeding tube port connector in a position to reduce patient discomfort due to feeding tube tension, the medical lanyard comprising:
a first strap assembly including:
a first loop sized to fit around a patient neck; and
an adjustment mechanism to adjust size of said first loop;
a second strap assembly including:
a second loop; and
an O-ring sized for the feeding tube port connector to fit within said O-ring, wherein said second loop extends through said O-ring;
a breakaway fastener for detachably connecting said second strap assembly to said first strap assembly, said breakaway fastener including:
a first portion in concert with said first loop; and
a second portion in concert with said second loop,
wherein said breakaway fastener is operable to detach said first portion and said second portion from each other upon an increased tension in said second strap assembly,
whereby when said second strap assembly is connected to said first strap assembly, said adjustment mechanism provides for adjusting the size of said first loop to vary length of the medical lanyard to locate said O-ring in proximity of a feeding tube entry point so that feeding tube tension is reduced.

2. The medical lanyard of claim 1, said breakaway fastener further comprising means for rotating said first portion and said second portion in relation to each other to provide a twisting operation between said first strap assembly and said second strap assembly.

3. The medical lanyard of claim 2, wherein said rotating means provides for lateral adjustment of said second strap assembly in relation to said first strap assembly.

4. The medical lanyard of claim 1, said first strap assembly further comprising a stop for preventing said adjustment mechanism from extending beyond said first loop.

5. The medical lanyard of claim 4, said stop further comprising at least one hole through which said first loop extends so that said first loop effectively serves as two substantially equivalent length straps.

6. The medical lanyard of claim 1, said first loop further comprising a single piece of material.

7. The medical lanyard of claim 6, wherein said first loop is one of the following: a cord, a rope, a string, a strap.

8. The medical lanyard of claim 1, said second loop further comprising a single piece of material.

9. The medical lanyard of claim 8, wherein said second loop is one of the following: a cord, a rope, a string, a strap.

10. The medical lanyard of claim 1, wherein said breakaway fastener is one of the following: a buckle, a cord, a clasp, a snap.

11. A medical lanyard for securing a feeding tube port connector in a position to reduce patient discomfort due to feeding tube tension, the medical lanyard comprising:
a clip assembly including:
a first loop; and
a clip attached to said first loop, said clip for securing said clip assembly to an item being worn by a patient;
an attachment assembly including:
a second loop; and
an O-ring sized for the feeding tube port connector to fit within said O-ring, wherein said second loop extends through said O-ring;
a breakaway fastener for detachably connecting said attachment assembly to said clip assembly, said breakaway fastener including:
a first portion in concert with said first loop; and
a second portion in concert with said second loop,
wherein said breakaway fastener is operable to detach said first portion and said second portion from each other upon an increased tension in said attachment assembly,
whereby when said attachment assembly is connected to said clip assembly, said clip provides for adjusting position of said clip assembly to vary position of the medical lanyard to locate said O-ring in proximity of a feeding tube entry point so that feeding tube tension is reduced.

12. The medical lanyard of claim 11, said breakaway fastener further comprising means for rotating said first portion and said second portion in relation to each other to provide a twisting operation between said first strap assembly and said second strap assembly.

13. The medical lanyard of claim 12, wherein said rotating means provides for lateral adjustment of said second strap assembly in relation to said first strap assembly.

14. The medical lanyard of claim 11, said first loop further comprising a single piece of material.

15. The medical lanyard of claim 14, wherein said first loop is one of the following: a cord, a rope, a string, a strap.

16. The medical lanyard of claim 11, said second loop further comprising a single piece of material.

17. The medical lanyard of claim 16, wherein said second loop is one of the following: a cord, a rope, a string, a strap.

18. The medical lanyard of claim 11, wherein said breakaway fastener is one of the following: a buckle, a cord, a clasp, a snap.

19. A medical lanyard for securing a feeding tube port connector in position to reduce patient discomfort due to feeding tube tension, the medical lanyard comprising:
a first strap assembly detachably connected to a second strap assembly via a fastener, said fastener including:
a first portion attached to said first strap assembly; and
a second portion attached to said second strap assembly,
wherein said fastener is operable for attaching and detaching said second strap assembly with said first strap assembly;
said first strap assembly further comprising:
a first strap connected to said first portion of said fastener, said first strap having a first length so that said first strap extends in a loop from said fastener to fit around a patient neck; and
an adjustment mechanism in concert with said loop of said first strap, said adjustment mechanism for sliding along said loop to partition said loop into a first loop portion and a second loop portion, wherein sliding said adjustment mechanism adjusts relative size of said first loop portion and said second loop portion; and
said second strap assembly further comprising:
a second strap connected to said second portion of said fastener; and
an O-ring, wherein said second strap extends through said O-ring, wherein opening size of said O-ring provides for the feeding tube port connector to fit securely within said O-ring,
whereby when said second strap assembly is attached to said first strap assembly, said adjustment mechanism provides for adjustment so that said O-ring is positioned in proximity of a feeding tube entry point into patient abdomen.

20. The medical lanyard of claim 19, said fastener further comprising a breakaway fastener.
